(19) United States
(12) Patent Application Publication
(10) Pub. No.: US 2013/0104899 A1
(43) Pub. Date: May 2, 2013

(54) RESPIRATOR AND ADJUSTMENT METHOD FOR SAME

(71) Applicant: Information-Management-Technology AG, Buchs (CH)

(72) Inventors: Harri Friberg, Mauren (LI); Jakob Daescher, Flaesch (CH)

(73) Assignee: INFORMATION- MANAGEMENT- TECHNOLOGY AG, Buchs (CH)

(21) Appl. No.: 13/723,571

(22) Filed: Dec. 21, 2012

Related U.S. Application Data
(63) Continuation-in-part of application No. 13/264,001, filed on Nov. 13, 2011, now abandoned.

(30) Foreign Application Priority Data
May 28, 2009 (CH) 0817/09

(51) Int. Cl. A61M 16/00; A61M 16/06; A61M 16/08

(52) U.S. Cl. CPC A61M 16/0057; A61M 16/0051; A61M 16/0875; A61M 16/06
USPC 128/204.23

(57) ABSTRACT

Respirators for respiration of patients are provided with interfaces 3 for input of at least one respiration parameter (PINSP, PEEP, RAM, I/E, RAT, T_{INSP}, T_{EXP}, T_{AZ}) that is relevant for the respiration. A respirator includes a computation unit for calculating a respiration curve (4, 4a, 4b) from the at least one respiration parameter (PINSP, PEEP, RAM, I/E, RAT, T_{INSP}, T_{EXP}, T_{AZ}), and includes a display unit 2 for displaying the calculated respiration curve (4, 4a, 4b). In addition, methods for operation of respirators are provided.

This application is a C-I-P continuation-in-part of copending U.S. application Ser. No. 13/264,001 that entered U.S. national phase with a section 371(c) date of Nov. 13, 2011 as a section 371 national-phase entry of PCT International application no. PCT/IB2010/052043 filed on May 10, 2010 and published as WO2010/136923A1 on Dec. 2, 2010, claiming benefit of priority to Swiss application no. 00817/09 filed on May 28, 2009; copending U.S. application Ser. No. 13/264,001 and prior PCT International application no. PCT/IB2010/052043 are expressly incorporated herein by reference, in their respective entireties and as to all their parts, for all intents and purposes, as if identically set forth in full herein.

### BACKGROUND

The invention relates to a respirator for artificial respiration of a patient, comprising an interface for input of at least one respiration parameter which is relevant for the respiration, a computation unit for calculating a respiration curve from the at least one respiration parameter and a display unit for displaying the calculated respiration curve. In addition, the invention relates to a method for adjusting a respirator for the respiration of a patient, such that at least one respiration parameter that is relevant for the respiration is input. Finally, the invention also relates to a related computer program product.

A respirator is an electrically or pneumatically operated machine for artificial respiration of persons whose own respiration is inadequate or has stopped. One criterion for adjusting respirators is the type of application. In invasive respiration the patient is either intubated or has had a tracheotomy. In noninvasive respiration, however, the patient is respirated through a tight-fitting mask. Another classification of respirators may be made according to emergency respirators, intensive care respirators and home respirators, on the basis of their field of use.

Essentially a distinction is made between volume-controlled, pressure-controlled, and time-controlled forms of respiration. In volume control, respiration or inspiration is continued, for example, until reaching a defined inspiration volume. Accordingly, in pressure-controlled respirators inspiration is continued until reaching a preset respiratory tract pressure. Time-controlled respirators, however, operate for a preset period of time. For control of respirators, a maximum pressure or a maximum volume, for example, is input, so that when this maximum is reached, the machine switches to the expiration phase. By varying the control parameters and the respiration parameters, many different ideal respiration curves may be set and thus many respiration techniques may be utilized for respiration therapy.

Therefore, different respiration parameters are used (e.g., respiration pressure, expiration pressure, the increase in pressure in inspiration, the ratio between inspiration time and expiration time, the set respiration rate, etc.), depending on the disease syndrome. To some extent, these settings may be made by the operator of a respirator under great tension or under hectic conditions, for example, when there is an acute situation. The economic pressure in the medical sector has also resulted in a decline in the funds available for training operators, so that even less-trained individuals are also operating respirators. All this increases the risk of mistakes in input that may lead to serious health consequences for a patient or, in the extreme case, may even result in death of the patient.

WO 02/058619, for example, also discloses a respirator that can calculate a respiration curve from certain parameters that have been input and can display it on a display device. Although this provides reasonable assistance for the operator, this respirator also presupposes a relatively great deal of technical knowledge and/or there is still a high risk of mistakes in input by operating personnel operating under stress.

### SUMMARY

An object of versions of the present invention is therefore to provide an improved respirator and/or an improved adjustment method, in particular a method in which operation is simplified.

This object may be achieved by a respirator having features disclosed herein, namely by a respirator of the type defined in the introduction in which one or more parameters from the following group are provided as respiration parameters: ideal respiration parameters, actual respiration parameters, or reference respiration parameters; and accordingly, one or more parameters from the following group are provided as the respiration curve: ideal respiration curve, actual respiration curve, or reference respiration curve.

In addition, the invention encompasses versions of method of type stated in the introduction, in which a respiration curve is calculated from the at least one respiration parameter, and the calculated respiration curve is displayed on a display unit.

Finally, the invention may be understood to encompass versions of computer program product having computer programs stored thereon, so as to be loaded into a memory of a respirator and execute the methods in which the computer program is executed in the respirator.

According to the invention, this results in the operator directly receiving a visual impression of the respiration curve that has been set. In addition, the effects of altered respiration parameters may be directly visible. This is a beneficial contribution because the risk of mistakes in input, and thus the risk of health impairment for the patient, may be greatly reduced.

In particular, ideal respiration parameters and an ideal respiration curve represent a desired respiration characteristic set on the machine, while actual respiration parameters and an actual respiration curve represent a respiration characteristic actually determined on a patient. Reference respiration parameters and a reference respiration curve represent an optimal respiration characteristic on a “normal patient.” For example, the inspiration pressure, the expiration pressure, the increase in pressure during inspiration, the ratio between inspiration time and expiration time and the respiration rate, for example, are also taken into account as respiration parameters. In comparison with known approaches, an important advantage therefore is that different types of respiration curves may be displayed. In other words, the respirator does not merely reflect the information input by the operator, even if it is in graphic form, but instead may also display an actual respiration characteristic that has been determined, and a stored or determined reference respiration characteristic in addition to the set ideal respiration characteristic. The reference respiration characteristic, in particular, provides valuable assistance for a less trained, uncertain, or stressed operator.

The computation unit in the respirator may be implemented in software and/or hardware. It is especially advantageous if a program which images the inventive method among other things and is stored in a memory is executed by a processor. The algorithm may be adapted especially easily to different conditions in this manner.

Advantageous embodiments and refinements of the invention are derived from the entirety of the present disclosure, as well as the description in conjunction with the drawings in the figures.

It is advantageous if the interface is provided for input of the at least one respiration parameter by evaluation of a drag-and-drop procedure performed by an operator on the respiration curve displayed on the screen. This allows a particularly intuitive adjustment of a respiration curve which may be performed reliably by an operator of the respirator even under high-stress conditions. A respiration curve in a certain range is selected and then modified as desired by dragging it. It is particularly advantageous if a touchscreen is used, because the curve may then be adjusted easily using a finger.

It is also advantageous if one or more of the following group of at least one patient parameter characterizing the patient is determined: ideal respiration parameter, ideal respiration curve, reference respiration parameter or reference respiration curve. As mentioned above, the reference respiration parameter and a reference respiration curve represent an optimal respiration behavior on a normal patient. This optimal respiration characteristic is, of course, different from one patient to the next, and depends on certain patient parameters. For example, age, weight, gender, general fitness and disease status may influence the aforementioned optimal respiration behavior. According to versions of the invention, optimal reference respiration parameters or an optimal reference respiration curve may be determined from at least one patient parameter, and may be offered to the operator of the respiration device as assistance. In an especially advantageous embodiment, ideal respiration parameters or an ideal respiration curve may be determined from the at least one patient parameter, so that the adjustment work is reduced to a minimum. For example, a key with which the reference respiration parameter is assigned to the ideal respiration parameters or the reference respiration curve is assigned to the ideal respiration curve may also be provided.

It is advantageous if the respirator effects a measuring of the actual respiration curve and/or the actual respiration parameters at an output of a respiration tube. The actual respiration characteristic prevailing with the patient may be ascertained in this way and then used as a basis for deriving additional treatment measures.

It is also advantageous if the respirator effects a measuring of the actual respiration curve and a determination of an actual respiration parameter from the actual respiration curve determined. Alternatively, it is also advantageous if the respirator effects a measuring of an actual respiration parameter and determines the actual respiration curve from the actual respiration parameter determined. In such variants, the actual respiration parameters are determined from a measured actual respiration curve, (i.e., the pressure or volume curve over time). For example, the inspiration pressure may then be determined very easily by forming the maximum value. However, the opposite path is also possible.

For example, an actual respiration curve may be calculated from measured actual respiration parameters. For example, the inspiration pressure, the expiration pressure, the inspiration time and the expiration time are measured for this purpose. In principle, the same algorithm which is also used for calculating the ideal respiration curve or the reference respiration curve may be used for this. However, the actual respiration parameters are input by using a measurement apparatus, whereas the ideal respiration parameters are input by using a keypad on the respirator, for example. However, the reference respiration parameters may be input via a table stored in the respirator or in a stored database.

It is also advantageous if the display unit is prepared for displaying at least one respiration parameter on the display unit. This is further support for the operator of the respirator, which also has a training effect. By displaying the respiration parameters, these parameters are also impressed upon the operator—even unconsciously—so that operation of the respirator may be accomplished more rapidly and reliably over time. Multiple respiration parameters may of course also be placed side by side. For example, the actual inspiration pressure may be displayed next to the ideal inspiration pressure, so that deviations between the two values are immediately visible.

In an advantageous variant of the invention, the respirator outputs a warning when the difference and/or the ratio between the two different respiration parameters exceeds a preselectable value. This automatically monitors whether the deviation between the actual inspiration pressure addressed above, for example, and the ideal inspiration pressure exceeds a predetermined threshold value. If this is the case, a visual warning and/or an acoustic warning is then output so as to draw the attention of the operator to this circumstance. The threshold value may be input by the operator himself, or may be provided at the factory.

In another advantageous variant of the present invention, the respirator determines the area between two different respiration curves during an observation period and outputs a warning when the area exceeds a predetermined value. This is another possibility for checking on whether the respiration is taking place in the desired manner or whether the ideal values that have been input are plausible with respect to the prevailing reference values. Not only are individual respiration parameters, for example, the expiration pressure, monitored here, but also the course of the respiration curve is monitored.

It is advantageous if the observation period is longer than the duration of one breath. For monitoring a respiration parameter and also for monitoring a respiration curve, an observation period which is longer than the duration of one breath is advantageously chosen. In this way, individual disturbances in the respiration process, for example, coughing by the patient or individual measurement errors, may be suppressed better. The observation period may be determined on the basis of a time indication or also on the basis of a number of breaths (e.g., 2.7 breaths).

Finally, it is advantageous if the respirator comprises a memory for permanent storage of a set of respiration parameters and/or a respiration curve. In this way, the respiration parameters are preserved, for example, even after the respirator has been shut down and thus may be retrieved conveniently, for example, when the same patient must be respirated repeatedly with pauses in between as part of a respiration therapy.

In conclusion, it is pointed out that the variants mentioned with respect to the inventive respirator and the resulting advantages are not based only on the respirator itself, but also on the inventive method. Those skilled in the art shall easily be able to adapt the teachings disclosed here to versions of the inventive method.

The above referred-to versions and refinements of the invention may be combined in any desired manner.

### BRIEF DESCRIPTION OF THE DRAWINGS

The present invention shall be explained in greater detail below on the basis of the exemplary embodiments depicted in the schematic drawings in the figures, in which:

FIG. 1 shows a schematic operating field of an exemplary respirator;

FIG. 2 shows an arrangement in which two respiration curves are shown instead of the respiration curve from FIG. 1;

FIG. 3 shows the area between two respiration curves;

FIG. 4 shows how one respiration curve can be modified with the help of a drag-and-drop procedure;

FIG. 5 depicts an exemplary respirator;

FIG. 6 depicts a first example of the inventive method;

FIG. 7 depicts an example of the inventive method, in which at least one first respiration parameter is measured;

FIG. 8 depicts an example of the inventive method, in which the first respiration curve is measured;

FIG. 9 depicts an example of the inventive method, in which respiration parameters are calculated based on a measured respiration curve;

FIG. 10 depicts an example of the inventive method with a drag-and-drop operation performed on a respiration curve;

FIG. 11 depicts an example of the inventive method, in which at least one respiration parameter is displayed;

FIG. 12 depicts an example of the inventive method, in which a difference between a first and a second respiration parameter is calculated;

FIG. 13 depicts an example of the inventive method, in which a ratio between a first and a second respiration parameter is calculated; and,

FIG. 14 depicts an example of the inventive method, in which a respiration parameter and/or a respiration curve is stored for later use.

### DETAILED DESCRIPTION

The same and similar parts are labeled with the same reference labels in the drawings in the figures, and elements and features having a similar function are provided with the same reference numerals but with different indices—unless otherwise indicated. Furthermore, any reference in this specification to “one embodiment,” “an embodiment,” “one version,” “a version,” “a variant,” and “one variant,” or similar recitation, should be understood to mean that a particular feature, structure, or characteristic described in connection with the version, variant, or embodiment is included in at least one such version, variant, or embodiment of the disclosure. Appearances of phrases “in one embodiment”, “in one version,” “in one variant,” and the like in various places in the specification are not necessarily all referring to the same variant, version, or embodiment, nor are separate or alternative versions, variants, or embodiments mutually exclusive of other versions, variants, or embodiments. Moreover, various features are described which may be exhibited by some versions, variants, or embodiments and not by others. Similarly, various requirements are described which may be requirements for some versions, variants, or embodiments but not others. Furthermore, as used throughout this specification, the terms ‘a’, ‘an’, ‘at least,’ ‘at least one’ do not denote a limitation of quantity, but rather denote the presence of at least one of the referenced item, and the term ‘a plurality’ denotes the presence of more than one referenced items.

FIG. 1 shows a schematic operating field 1 of an exemplary respirator including a display unit 2 and several operating keys 3. A respiration curve 4 is shown on the display unit 2. In addition, an area 5 in which respiration parameters are displayed is shown on the display unit 2.

In a first example, ideal respiration parameters are input by the operator of the respirator via the operating keys 3 of the respirator. The following parameters are fixedly assigned to the operating keys in this example: inspiration pressure PINSP, expiration pressure PEEP, the increase in pressure in inspiration RAM, the ratio between inspiration time and expiration time I/E and the respiration rate RAT. FIG. 1 also shows the importance of the inspiration pressure PINSP, the expiration pressure PEEP, the increase in the pressure in inspiration RAM, the inspiration time T_{INSP} and the expiration time T_{EXP} (note: this information is not necessarily also displayed on a display unit 2 and serves here primarily to provide a better understanding of the invention). The ratio between the inspiration time T_{INSP} and the expiration time T_{EXP} now corresponds to I/E, the sum of the two times 1/respiration rate RAT.

According to the prior art, an operator must know in detail the effects of the individual parameters on respiration, which often leads to mistakes in operation, especially with the stress associated with emergency situations. The inventive method then calculates an ideal respiration curve 4 for the input parameters and displays this on the display unit 2. This is an enormous assistance for the operator, because the effect of a certain input is immediately shown visually. In an advantageous variant of the invention, the parameters themselves are also displayed in an area 5. The operator may thus become more easily familiar with such conventional parameter sets and may adjust the respirator more rapidly the next time accordingly.

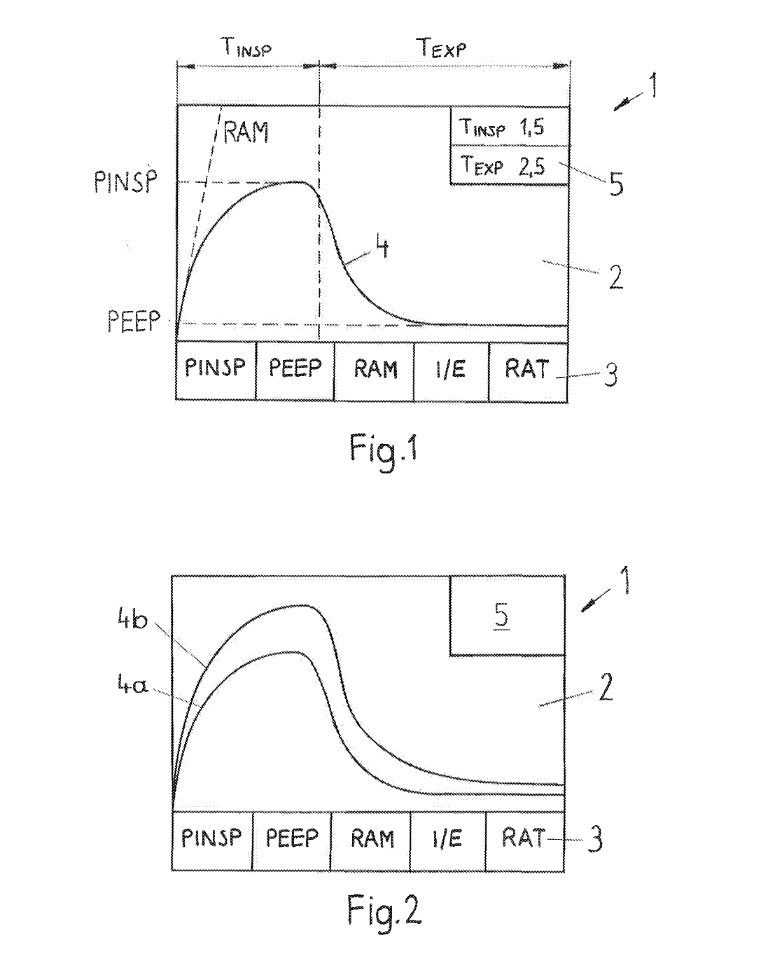

FIG. 2 shows an arrangement which is very similar to the arrangement in FIG. 1. However, instead of the respiration curve 4, two respiration curves 4a and 4b are shown here.

In a first example, the respiration curve 4a is a reference respiration curve, while respiration curve 4b is an ideal respiration curve. In this example, the operator of the respirator inputs patient parameters, which characterize the patient. These may include, for example, weight, age, gender, severity of illness, etc. The respirator then calculates a reference respiration curve 4a from the patient parameters and displays this on the display unit 2. In the next step, the operator inputs ideal respiration parameters for the patient. The respirator calculates an ideal respiration curve 4b from these parameters as already mentioned above, and also displays this on the display unit 2. Then the operator sees not only the effects of the various ideal respiration parameters on the basis of the ideal respiration curve 4b, but also a deviation from the reference respiration curve 4a. In this way, the operator may “approach” an ideal reference respiration curve 4a or may also deviate from it intentionally because, for example, there may be medical reasons against the use of a reference respiration curve. In an advantageous variant, the reference respiration parameters may be assigned automatically to the ideal respiration parameters and/or the reference respiration curve may be assigned automatically to the ideal respiration curve, so that the adjustment work is facilitated. This step may also be performed automatically after input of the patient parameters.

In a second example, the respiration curve 4a is an actual respiration curve while the respiration curve 4b is, in turn, an ideal respiration curve. In this constellation, the operator of the respirator may check on the extent to which the actual respiration curve 4a measured on the patient corresponds to the set ideal respiration curve 4b and may initiate countermeasures accordingly as needed, that is, when there is a great deviation.

In a third example, the respiration curve 4a is an actual respiration curve but the respiration curve 4b is a reference respiration curve. In this variant, the operator of the respirator may check on the extent to which the actual respiration curve 4a measured on the patients corresponds to a reference respiration curve 4b, which is calculated on the basis of at least one patient parameter characterizing the patient. In this way, the operator may ascertain the extent to which the actual respiration curve 4a deviates from the “norm.”

In the examples given above, actual respiration curve is always measured, that is, a curve of pressure over time was measured. However, a variant in which the actual respiration parameters determined are merely input, that is, measured, and an actual respiration curve is calculated from them is also conceivable. In this case, for example, the inspiration pressure (i.e., the maximum pressure during the respiration process), the expiration pressure PEEP (i.e., the minimum pressure during the respiration process), the inspiration time (time between the increase in pressure and the drop in pressure) and the expiration time (time between the pressure drop and the pressure rise) may be measured and an actual respiration curve calculated from them. However, actual respiration parameters may of course also be determined from the actual respiration curve. For example, the inspiration pressure PINSP can be determined by evaluating the actual respiration curve (that is, determining its maximum value).

In another advantageous embodiment of the invention, various respiration parameters may be compared with one another in the area 5 of the display unit 2. For example, the reference respiration parameters may be compared with the ideal respiration parameters (Example 1), the actual respiration parameters may be compared with the ideal respiration parameters (Example 2) or the actual respiration parameters may be compared with the reference respiration parameters (Example 3). In the upper right corner of the display unit, a corresponding table is advantageously inserted (note: however, no concrete values are shown in area 5 in this example).

In an advantageous variant of the invention, an ideal respiration curve, a reference respiration curve and an actual respiration curve and/or ideal respiration parameters, reference respiration parameters and actual respiration parameters may of course also be displayed at the same time.

In another advantageous variant of the invention, a warning may be output when the difference, that is, the ratio between two different respiration parameters exceeds a predetermined value.

For example, a deviation of the actual inspiration pressure from the ideal inspiration pressure or a deviation in the ideal respiration rate from the reference respiration rate may thus be ascertained, and the operator of the respirator may be thereby notified. Although an optical display is sufficient in the case of a deviation in a ideal respiration parameter from a reference respiration parameter (it is assumed that the operator has turned his/her attention to the display unit 2 while setting the respirator), preference should be given to an (additional) acoustic warning in the case of a deviation of an actual respiration parameter from an ideal or reference respiration parameter.

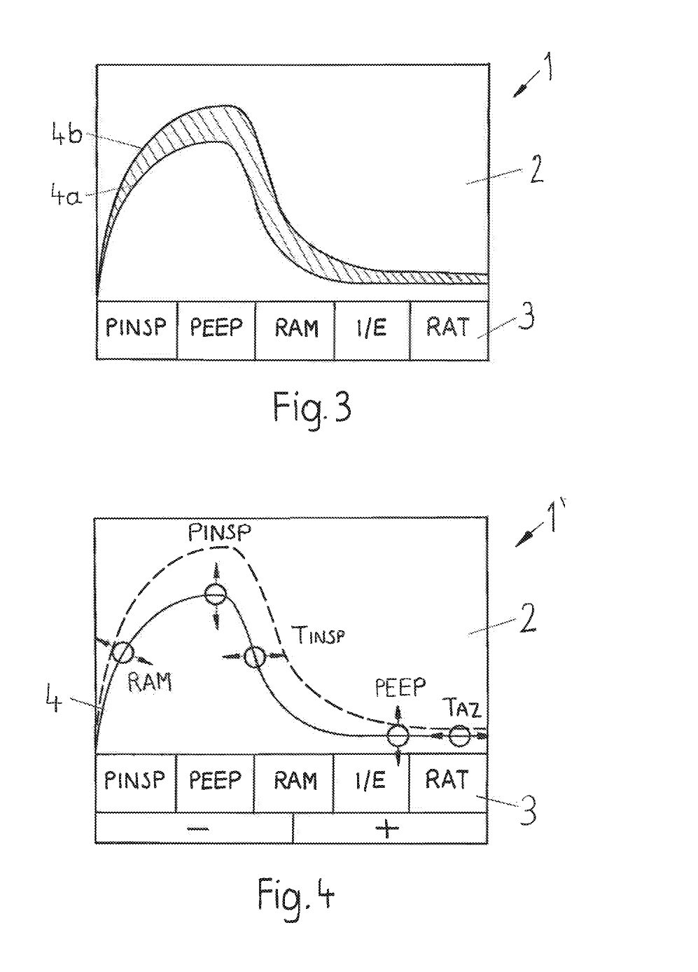

Similarly, the area between two different respiration curves may be used for output of a warning during an observation period. For example, the area between the actual respiration curve and the ideal respiration curve or between the ideal respiration curve and the reference respiration curve may be determined and a warning may be output when the difference exceeds a certain measure (in this regard, see the hatched area in FIG. 3).

For monitoring a respiration parameter and also for monitoring a respiration curve, an observation period which is longer than the duration of one breath is advantageously used. In this way, individual disturbances in the respiration process, e.g., coughing by the patient or individual measurement errors, can be suppressed better. The observation period may be determined on the basis of a time specification or on the basis of a number of breaths (e.g., 2.7 breaths).

In another variant respiration parameters and/or respiration curves and/or patient parameters may also be stored permanently. These are then preserved, for example, even after the respirator has been turned off and can thus be retrieved again conveniently, for example, if the same patient must be respirated repeatedly with pauses in between.

In the previous examples and variants, it has been assumed that the respiration parameters may be set by keys 3, buttons, regulators and the like provided on the machine. In an advantageous variant, these input elements are arranged on a touchscreen. So-called “soft keys” may be implemented in this way; these are adjustment elements which can change the function assigned to them. For example, the same keys may be provided for input of respiration parameters or patient parameters, depending on the set function.

In a particularly advantageous variant, the respiration curve may be modified directly, that is, without a change in a certain respiration parameter. To do so, a respiration curve and/or an area of same is selected on the touchscreen and modified by dragging it. This method is best known by the term “drag-and-drop.” Operation of a respirator thus becomes more intuitive. FIG. 4 shows examples of points on a respiration curve 4, where the curve may be “captured” by using a finger and then dragged. The arrows symbolize the direction in which dragging is possible, for example, producing a change in the respiration curve 4. The values of the respective respiration parameters are of course continuously adapted to the new respiration curve 4 and displayed. Use of a drag-and-drop procedure is of course not limited to use of a touchscreen, but may also be performed in conjunction with other input devices, for example, in combination with keys, levers, or a computer mouse. The arrangement of the areas where the respiration curve 4 may be captured are also to be seen as mere examples. In particular, for the change in the ratio between the inspiration time and the expiration time I/E and the respiration rate RAT and/or respiration time T_{AZ}, areas may also be provided at the side of the respiration curve 4, for example, at the upper or lower edges of the display screen.

FIG. 4 also shows additional plus and minus keys with the help of which the value of a previously selected respiration parameter may be altered (for example, with the keys 3 from FIGS. 1 to 3, the value may be increased by pressing in the upper/right area of the key 3 or the value may be lowered by pressing in the lower/left area of the key 3).

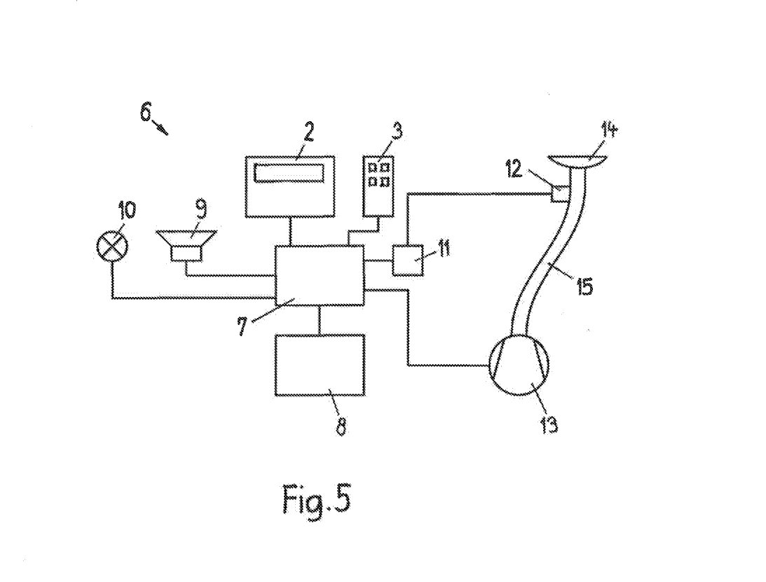

FIG. 5 depicts an exemplary respirator 6, comprising a computation unit 7, a display unit 2, an interface respectively operating keys 3, a memory 8, a speaker 9, an alarm light 10, a measuring unit 11, a sensor 12, a compressor 13, a face mask 14 and a tube 15. In this example the computation unit 7 is connected to the display unit 2, the interface/operating keys 3, the memory 8, the speaker 9, the alarm light 10, the measuring unit 11 and the compressor 13. The compressor 13 is connected to the face mask 14 via the tube 15. Finally, the sensor 12 is connected to the measuring unit 11.

The function of the respirator 6 is as follows:

The computation unit 7 controls the compressor 13 so as to ventilate a patient in a predetermined way. For this reason, the computation unit 7 may also comprise a motor controller module. Alternatively, a dedicated motor controller may be arranged between the computation unit 7 and the compressor 13. Compressed air is guided by the tube 15 to the face mask 14 worn by the patient.

During operation, at least one respiration parameter is selected/input from at least one of the group of respiration parameters consisting of PINSP, PEEP, RAM, I/E, RAT, T_{INSP}, T_{EXP}, and T_{AZ} via the interface/operating keys 3. Then the computation unit 7 calculates, from said at least one respiration parameter, a first respiration curve selected from the group of respiration curves consisting of ideal respiration curve, actual respiration curve, and reference respiration curve.

Additionally, the computation unit 7 calculates a second respiration curve selected from the group of respiration curves consisting of ideal respiration curve and reference respiration curve, wherein said second respiration curve is not identical to said first respiration curve. At least one of said first and second respiration curves is shown on the display unit 2. Moreover, the computation unit 7 determines the area between said first respiration curve and said second respiration curve over a selected observation period and signals a warning when said area exceeds a predetermined value. This warning may be output on the display unit 2 and/or via the speaker 9 and/or by the alarm light 10.

In a preferred version a set of respiration parameters and/or a respiration curve may be stored in memory 8 for later use. Accordingly, a patient may be ventilated repeatedly with pauses in between as part of a respiration therapy, in a convenient way.

The display 2 may be embodied as an LCD-screen (liquid crystal display) as a TFT-screen (Thin-film transistor), OLED-display (organic light emitting diode) or any other suitable graphical display. Moreover, touch screens, for example resistive touch screens or capacitive touch screens, may be used. Such touch screens (e.g., a Hitachi 530s touch screen) provide comfortable handling of the inventive respirator 6 and in particular provide drag-and-drop operation.

The computation unit 7 may be any future or existing processor, including, but not limited to, an Intel® processor(s) or AMD® processor(s), preferably Microsoft® compatible, like for example, Intel® Pentium® processors.

The machine-readable memory 8 may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, MultiMedia Cards (MMCs), secure digital (SD) cards, such as miniSD and microSD cards, or other type of media/machine-readable medium suitable for storing electronic instructions.

In the case that an actual respiration curve has to be displayed and/or evaluated, an actual respiration curve and/or actual respiration parameters may be acquired by sensor 12. The sensor 12 may provide a signal, that may directly be processed by the computation unit 7 or that is preprocessed by the measuring unit 11 as shown in FIG. 5. The measuring unit 11 may include a sensor and a control processor to which the sensor is connected. The sensor of the measuring unit 11 may be any future or existing sensor, including but not limited to, a sensor connected or comprising an analog-digital-converter (ADC), preferably a 12-bit-8-channel converter. The control processor of the measuring unit 11 be any future or existing processor(s), including, but not limited to, a Motorola® processor(s) or ARM® processor(s), preferably a Motorola® Coldfire®. If an actual respiration curve is acquired by the sensor 12 and the measuring unit 11, the computation unit 7 may calculate actual respiration parameters thereof. However, the computation unit 7 may also calculate an actual respiration curve based on actual respiration parameters acquired by the sensor 12 and the measuring unit 11.

For example the sensor 12 can acquire the inspiration pressure PINSP and the expiration pressure PEEP corresponding to the maximum value of the pressure and its minimum value. Together with a timer, which may be integrated in the measuring unit 11 or the computation unit 7 also the inspiration time T_{INSP}, the expiration time T_{EXP} and the increase in pressure in inspiration RAM can be determined. In principle, the same algorithm may be used for the generation of a respiration curve, which is also used for calculating a respiration curve based on parameters input via the interface/operating keys 3. Instead of the pressure sensor 12 or in addition thereto, also a flow sensor 12 may be provided.

It should be noted that the pressure need not necessarily be measured nearby the face mask 14. By contrast, the sensor 12 may also be arranged nearby the compressor 13 so as to save wiring.

Embodiments of the present invention include various steps that shall be described below. The steps may be performed by hardware components or may be embodied in machine-executable instructions, which may be used to cause a general purpose or special-purpose processor programmed with the instructions to perform the steps. Alternatively, the steps may be performed by a combination of hardware, software, firmware and/or by the intervention of human operators.

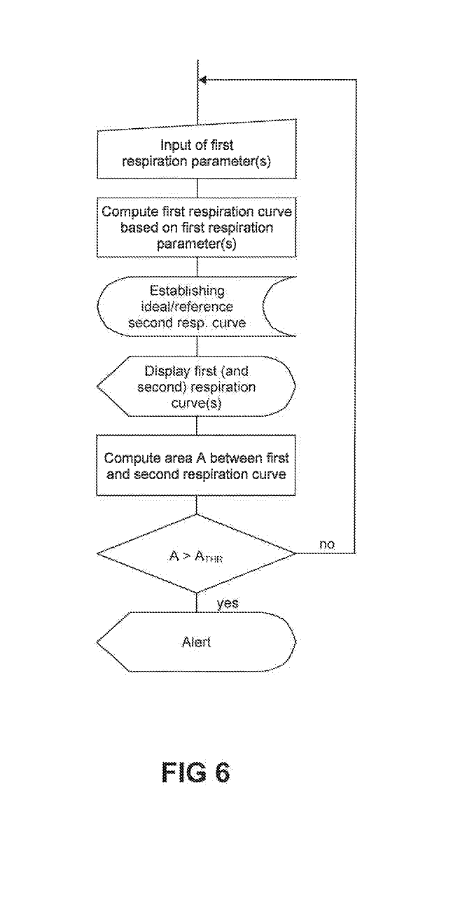

FIG. 6 depicts a first example of the inventive method. In a first step, at least one respiration parameter is input into an interface 3. In a second step, a first respiration curve is calculated based on the at least one respiration parameter in the computation unit 7. Next, a second respiration curve, which may be an ideal respiration curve or reference respiration curve for example, is established. Then, the first respiration curve is displayed on a display 2. Optionally, also the second curve may be displayed on the display 2. In a further step, the area A between first and second respiration curve is computed in the computation unit 7. If said area A exceeds a predetermined threshold value A_{THR}, an alarm is output, for example via the speaker 9 and/or the alarm light 10.

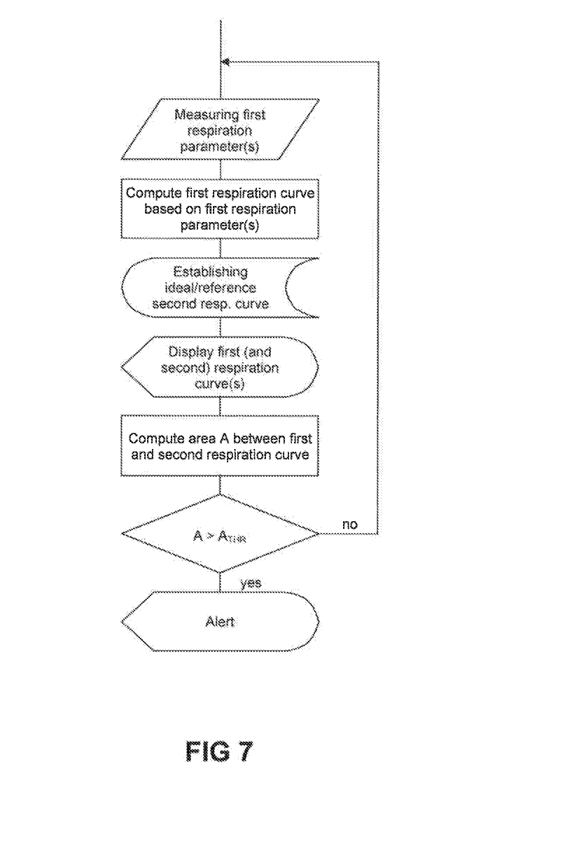

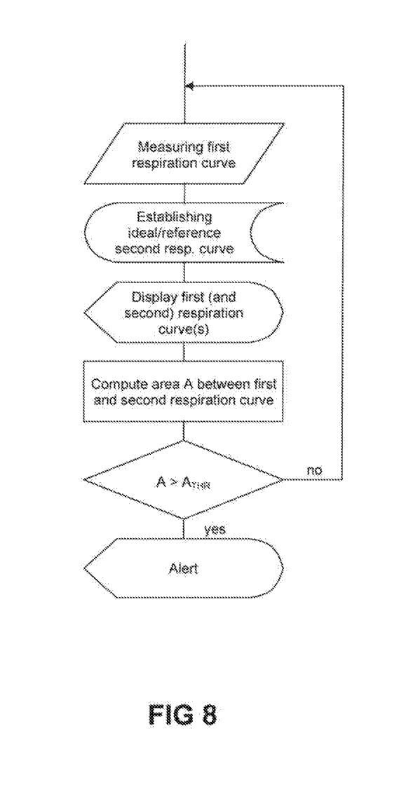

FIG. 7 depicts a method, which is similar to the method shown in FIG. 6. In contrast, in FIG. 8 the at least one respiration parameter is not input via the interface 3 but measured by the sensor 12.

FIG. 8 depicts another method, which is similar to the method shown in FIG. 7. In contrast, the first respiration curve is directly measured by the sensor 12. Accordingly, the step of computation of a first respiration curve based on at least first respiration parameter may be omitted.

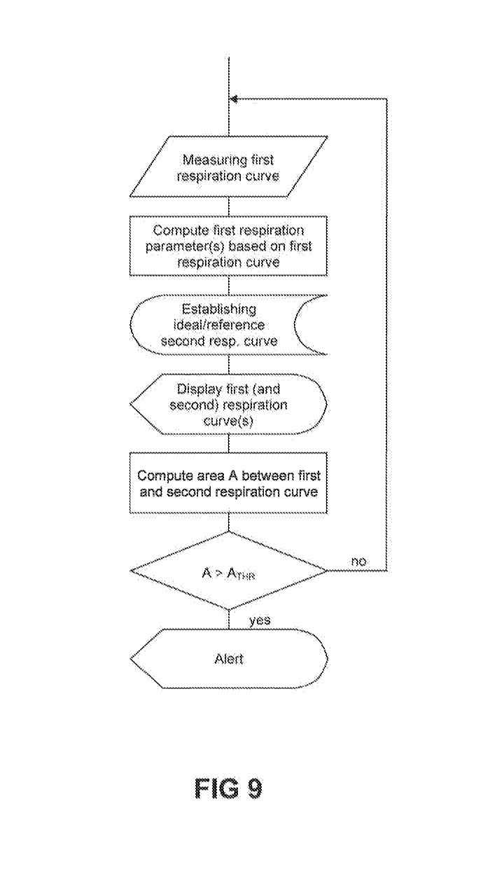

FIG. 9 depicts a method, which is similar to the method shown in FIG. 8. In contrast, in FIG. 9 at least one first respiration parameter is computed based on the measured first respiration curve.

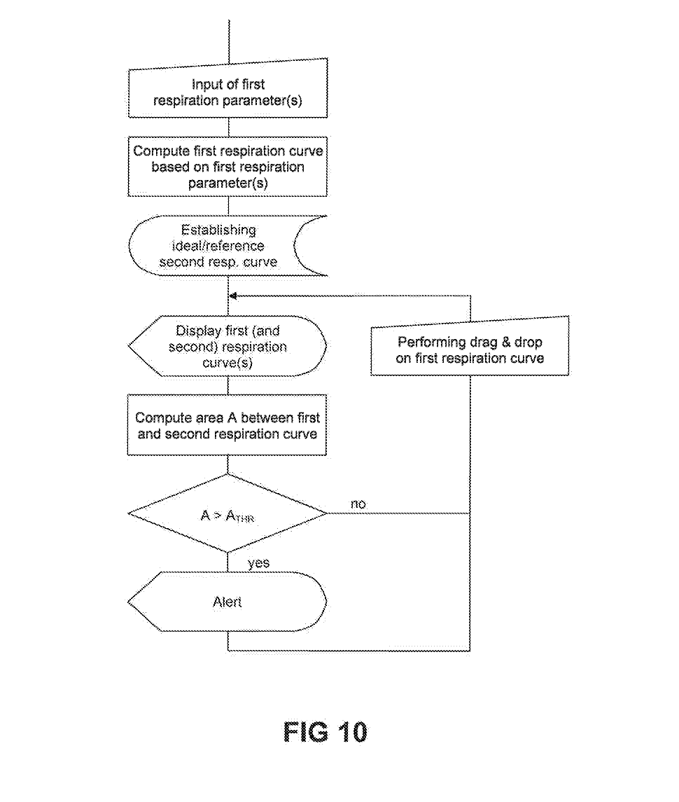

FIG. 10 depicts an example that is similar to the method presented in FIG. 6 and in which the first respiration curve may recursively be changed by a drag-and-drop operation. The display is continuously updated and also the area A between the first and the second respiration curve is continuously compared to the predetermined threshold value A_{THR}.

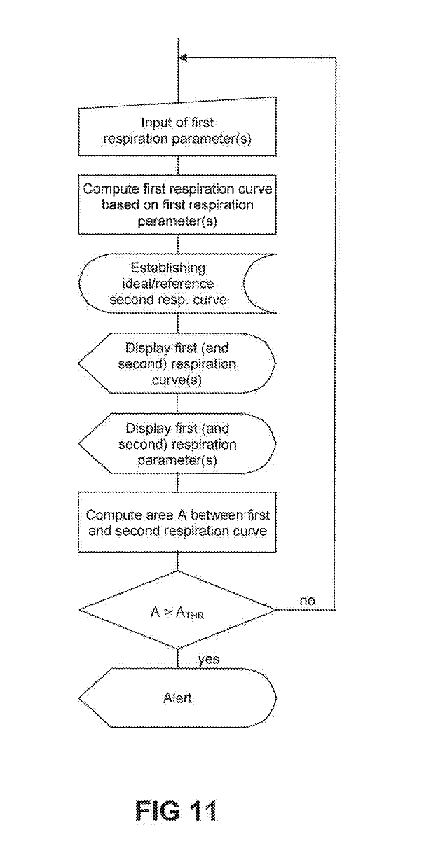

FIG. 11 depicts another example, which is similar to the method presented in FIG. 6. In contrast, in FIG. 11 the first respiration parameter(s) are displayed in addition to the respiration curves on the display 2. Optionally, also at least one second respiration parameter may be displayed.

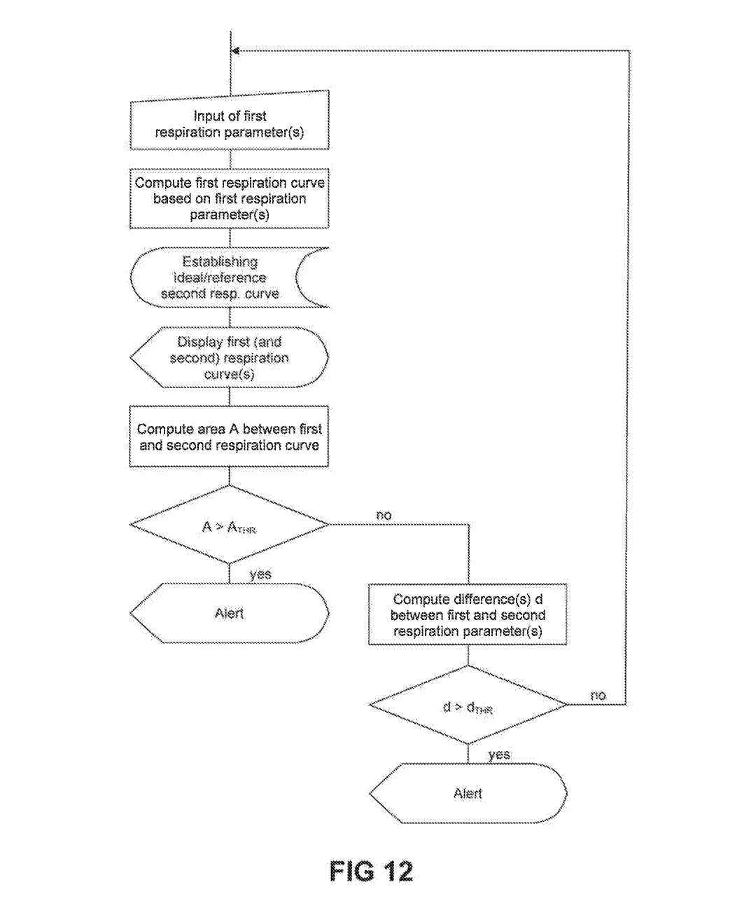

FIG. 12 depicts yet another example, which is similar to the method presented in FIG. 6. In contrast, in FIG. 12 not just the area A between the first and the second respiration curve is compared to the predetermined threshold value A_{THR}, but also a difference d between at least one first respiration parameter and at least one second respiration parameter is compared to a predetermined threshold value d_{THR}. If the difference d exceeds this threshold value d_{THR}, an alarm is output.

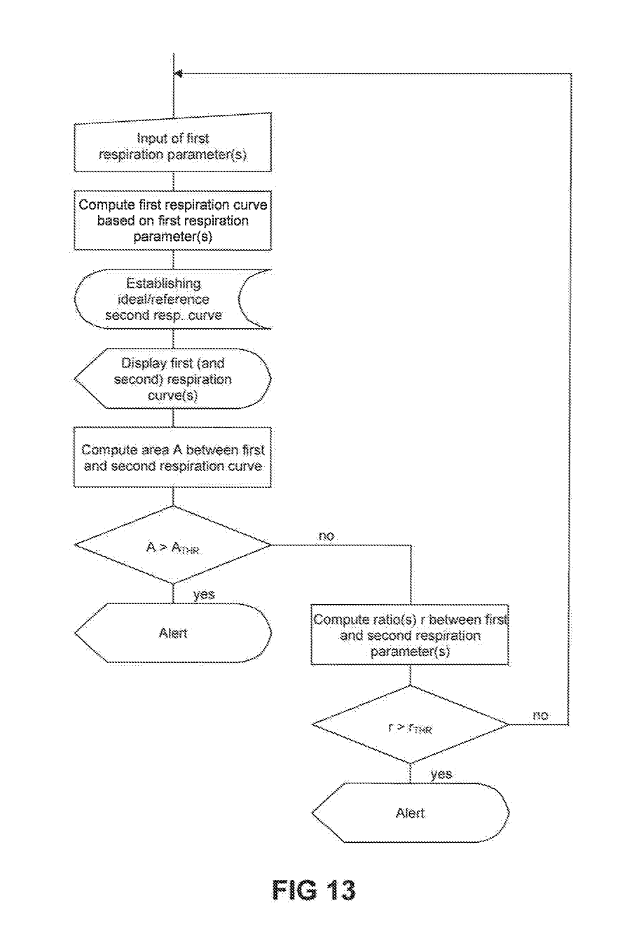

FIG. 13 depicts an example that is quite similar to the one shown in FIG. 12. In contrast, in FIG. 13 instead of the difference(s) between first and second parameter(s), the ratio(s) between first and second parameter(s) are compared to a threshold value r_{THR}. If the ratio r exceeds this threshold value r_{THR}, again an alarm is output.

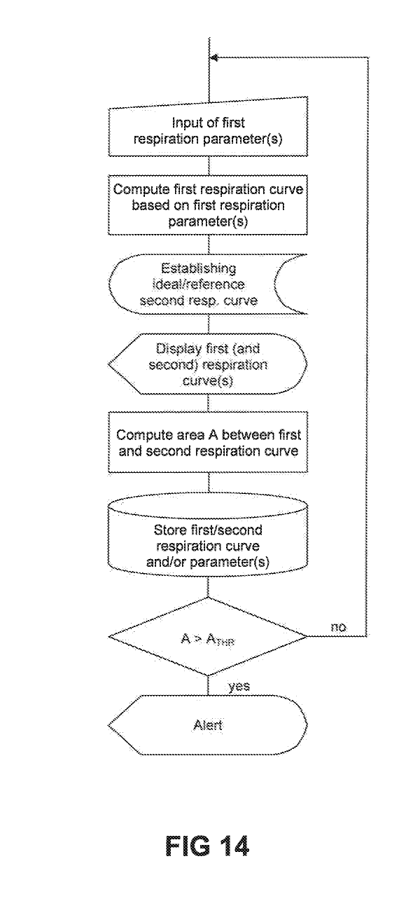

Finally, FIG. 14 depicts another example that is similar to the method shown in FIG. 6. In contrast, in FIG. 14 the first and/or second respiration curves and/or the first and/or second respiration parameter(s) may be stored in memory 8 for later use.

Versions of the present invention may be provided as a computer program product which may include a machine-readable medium having stored thereon instructions which may be used to program a processor associated with a ventilation control system to perform various processing. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, MultiMedia Cards (MMCs), secure digital (SD) cards, such as miniSD and microSD cards, or other type of media/machine-readable medium suitable for storing electronic instructions. Moreover, versions of the present invention may also be downloaded as a computer program product. The computer program may be transferred from a remote computer to a requesting computer by way of data signals embodied in a carrier wave or other propagation medium via a communication link (e.g., a modem or network connection). For example, various subsets of the functionality described herein may be provided within a legacy or upgradable ventilation system as a result of installation of a software option, or via performance of a firmware upgrade.

In conclusion, it is pointed out that the various exemplary embodiments depicted in the figures may also form the basis for independent inventions.

In addition, it is noted that the invention of course refers not only to pressure-controlled respirators but also to volume-controlled or time-controlled respirators, although this may not depict explicitly in the figures. Those skilled in the art shall be able to easily apply the teachings disclosed herein to the aforementioned fields, in particular by employing different respiration parameters, such as for example the inspiration volume. Thus, in closing, it should be noted that the invention is not limited to the abovementioned versions and exemplary working examples. Further developments, modifications and combinations are also within the scope of the patent claims and are placed in the possession of the person skilled in the art from the above disclosure. Accordingly, the techniques and structures described and illustrated herein should be understood to be illustrative and exemplary, and not necessarily limiting upon the scope of the present invention. The scope of the present invention is defined by the appended claims, including known equivalents and unforeseeable equivalents at the time of filing of this application.

### LIST OF REFERENCE LABELS

1, 1′ operating field
2 display unit
3 operating keys
4, 4a, 4b respiration curve
5 area for respiration parameters
6 respirator
7 computation unit
8 memory
9 speaker
10 alarm light
11 measuring unit
12 sensor
13 compressor
14 face mask
15 tube
A_{THR} area threshold value
d_{THR} difference threshold value
r_{THR} ratio threshold value
I/E ratio between inspiration time and expiration time
PEEP expiration pressure
PINSP inspiration pressure
RAM increase in pressure in inspiration
RAT respiration rate
T_{AZ} respiration time
T_{EXP} expiration time
T_{INSP} inspiration time

What is claimed is:

1. A respirator comprising:
an interface configured to receive input of at least one respiration parameter selected from at least one of the group of respiration parameters consisting of PINSP, PEEP, RAM, I/E, RAT, T_{INSP}, T_{EXP}, and T_{AZ};
a computation unit configured to calculate, from said at least one respiration parameter, a first respiration curve selected from the group of respiration curves consisting of ideal respiration curve, actual respiration curve, and reference respiration curve, said computation unit being in operative communication with said interface;
said computation unit calculating a second respiration curve selected from the group of respiration curves consisting of ideal respiration curve and reference respiration curve, wherein said second respiration curve is not identical to said first respiration curve;
said computation unit determining the area between said first respiration curve and said second respiration curve over a selected observation period, said computation unit signaling a warning when said area exceeds a predetermined value; and,
a display unit configured to display at least one of said first and second respiration curves, said display unit being in operative communication with said computation unit.

2. The respirator as claimed in claim 1, wherein:
said interface is a drag-and-drop interface.

3. The respirator as claimed in claim 1, wherein:
said computation unit determines, from at least one characterizing patient parameter, at least one patient parameter from at least one of the group of parameters consisting of ideal respiration parameter, ideal respiration pressure curve, reference respiration parameter, and reference respiration pressure curve.

4. A respirator as claimed in claim 1, further comprising:
at least one sensor for measuring the actual respiration pressure curve.

5. A respirator as claimed in claim 1, further comprising:
at least one sensor for measuring an actual respiration pressure parameter.

6. A respirator as claimed in claim 1, further comprising:
at least one sensor for measuring the actual respiration pressure curve as a determinant of at least one actual respiration parameter.

7. A respirator as claimed in claim 1, further comprising:
at least one sensor for measuring at least one actual respiration parameter as a determinant of the actual respiration pressure curve.

8. The respirator as claimed in claim 1, wherein:
said display unit is configured to display at least one respiration parameter.

9. The respirator as claimed in claim 1, wherein:
said computation unit signals a warning when the difference between two different respiration parameters exceeds a predetermined value.

10. The respirator as claimed in claim 1, wherein:
said computation unit signals a warning when the ratio between two different respiration parameters exceeds a predetermined value.

11. A respirator as claimed in claim 1, further comprising:
a memory apparatus configured to permanently store respiration data selected from the group consisting of a set of respiration parameters, a respiration pressure curve, and a set of respiration parameters with a respiration pressure curve.

12. A method for adjusting a respirator comprising the steps of:
inputting at least one respiration parameter into an interface;
calculating a first respiration curve from the at least one respiration parameter;
establishing a second respiration pressure curve;
displaying the first respiration pressure curve on a display;
determining the area between the first and second respiration pressure curves during an observation period; and,
outputting a warning when the area exceeds a predetermined value.

13. A method for adjusting a respirator as claimed in claim 12, further comprising the steps of:
evaluating a drag-and-drop on the first respiration pressure curve displayed on the display; and,
inputting at least one respiration parameter based on said step of evaluating.

14. A method for adjusting a respirator as claimed in claim 12, further comprising the steps of:
providing at least one respiration parameter selected from at least one of the group of respiration parameters consisting of PINSP, PEEP, RAM, I/E, RAT, T_{INSP}, T_{EXP}, and T_{AZ}; and,
providing at least one respiration pressure curve selected from at least one of the group of curves consisting of ideal respiration pressure curve, actual respiration pressure curve, and reference respiration pressure curve.

15. A method for adjusting a respirator as claimed in claim 14, further comprising the step of:
determining at least one patient parameter selected from at least one of the group of parameters consisting of ideal respiration parameter, ideal respiration pressure curve, reference breathing parameter, and reference respiration pressure curve.

16. A method for adjusting a respirator as claimed in claim 14, further comprising the step of:
measuring at an output of a respirator tube the actual respiration pressure curve, or an actual respiration pressure parameter, or both the actual respiration pressure curve and an actual respiration pressure parameter.

17. A method for adjusting a respirator as claimed in claim 16, further comprising the steps of:
measuring the actual respiration pressure curve; and,
determining the actual respiration parameter from the measured actual respiration pressure curve.

18. A method for adjusting a respirator as claimed in claim 16, further comprising the steps of:
measuring the actual respiration parameter; and,
determining the actual respiration pressure curve from the measured actual respiration parameter.

19. A method for adjusting a respirator as claimed in claim 12, further comprising the step of:
displaying at least one respiration parameter on the display.

20. A method for adjusting a respirator as claimed in claim 12, further comprising the step of:
outputting a warning when the difference between two different respiration parameters exceeds a predetermined value.

21. A method for adjusting a respirator as claimed in claim 12, further comprising the step of:
outputting a warning when the ratio between two different respiration parameters exceeds a predetermined value.

22. A method for adjusting a respirator as claimed in claim 12, further comprising the step of:
permanently storing respiration data including a set of respiration parameters, or at least one respiration pressure curve, or a set of respiration parameters with at least one respiration pressure curve.
